(19) United States
(12) Patent Application Publication
(10) Pub. No.: US 2010/0055032 A1
(43) Pub. Date: Mar. 4, 2010

(54) METHODS FOR VASCULAR IMAGING USING NANOPARTICULATE CONTRAST AGENTS

(75) Inventors: Reinhard Koenig, New Hope, PA (US); Taffy J. Williams, Lansdale, PA (US)

Correspondence Address: FISH & RICHARDSON P.C., P.O. BOX 1022, MINNEAPOLIS, MN, 55440-1022, US

(73) Assignee: NanoScan Imaging LLC, Lansdale, PA (US)

(21) Appl. No.: 12/487,349

(22) Filed: Jun. 18, 2009

Related U.S. Application Data
(63) Continuation of application No. 10/290,582, filed on Nov. 7, 2002, now abandoned.
(60) Provisional application No. 60/346,162, filed on Nov. 7, 2001.
(60) Provisional application No. 60/346,519, filed on Jan. 8, 2002.

(51) Int. Cl. A61K 51/00; A61K 49/00; A61K 49/04

(52) U.S. Cl. 424/1.11; 424/9.1; 424/9.4; 424/9.3; 977/927; 977/928; 977/930; 977/929; 977/773

(57) ABSTRACT

New and sensitive methods for imaging the perfusion of tissues and the extravasation of blood out vessels have been developed. The present invention is useful in the imaging of microperfusion in organ tissues (e.g., heart, liver, brain and kidneys) to aid in evaluating the perfusion status of organs on the level of the smallest blood vessels (i.e. capillaries). The present invention also provides methods and compositions for imaging and evaluating macrophages and plaque, e.g., vulnerable plaque. Such evaluations are important in a number of clinical diagnoses, including assessing organ damage associated with angina pectoris, heart attack, stroke, and the like, as well as assessing vessel leakages associated with aneurisms, diffuse bleedings after trauma, and the like.

### RELATED APPLICATIONS

This application claims priority to U.S. Provisional Application No. 60/346,162 entitled “Method for Vascular Imaging Using Nanoparticulate Contrast Agents,” filed on Nov. 7, 2001, and U.S. Provisional Application No. 60/346,519, entitled “Method for Vascular Imaging Using Nanoparticulate Contrast Agents,” filed on Jan. 8, 2002, the contents of which are incorporated herein by reference.

### BACKGROUND OF THE INVENTION

Coronary heart disease (or coronary artery disease (CAD)) is the leading cause of death in the United States for men and women. According to the American Heart Association, about every 29 seconds someone in the U.S. suffers from a coronary heart disease-related event and about every minute someone dies from such event. The lifetime risk of having coronary heart disease after age 40 is 49% for men and 32% for women. As women get older, the percentage increases almost to that of men. Although mortality from heart disease has declined steadily over the past three decades in the U.S., the total burden of coronary disease is predicted to increase substantially over the next 30 years due to the increasing size of the elderly population. The cost of medical care and lost economic productivity due to heart disease in the U.S. was estimated to exceed $60 billion in 1995.

There are many factors that increase the risk for coronary heart disease. Some of the risks are based on family history (i.e., genetics) while others are more controllable. Such risk factors include family history of coronary heart disease (especially before the age of 50), male gender, age ≧65, tobacco smoking, high blood pressure, diabetes, high cholesterol levels (specifically, high low density lipoprotein [LDL] cholesterol levels and low high density lipoprotein [HDL] cholesterol levels), lack of physical activity, obesity, high blood homocysteine levels and post-menopause in women. Other factors, including infections that cause inflammatory responses within the arterial wall, are currently being investigated. Interestingly, recent studies have shown that the activation of macrophages (phagocytic white blood cells involved in the removal of foreign material from within body tissues) located in the inner walls of the coronary arteries may play an important role in the formation of coronary plaques. It has further been shown that macrophages have to ability to migrate to areas of inflammation and deposits of foreign material, such as vascular plaques.

Pathologically, coronary heart disease is characterized by the narrowing of the small blood vessels that supply blood and oxygen to the heart. Coronary heart disease usually results from the build up of fatty material and plaque (atherosclerosis). This material is associated with fibrous connective tissue and frequently includes deposits of calcium salts and other residual material. The damage caused by coronary heart disease varies. As the arteries narrow, the flow of blood to the heart can slow or stop, resulting in symptoms such as cause chest pains (stable angina), shortness of breath, or a heart attack (i.e. myocardial infarction). Thrombus formation may also result in the roughened areas, which results from plaque build-up.

Of considerable concern is ‘vulnerable’ or ‘active’ plaque which has the tendency to break away from the vessel and be deposited in a narrow vessel, often leading to coronary heart disease and atherosclerosis. If loosened, this material could travel through the vascular system causing a coronary attack, a stroke if in the region of the brain, or an occlusion of a vessel if in the leg. Relief of focal high-grade obstruction may control symptoms, but the patient usually is left with numerous nonobstructive plaques prone to later rupture and cause infarction.

While conventional imaging and detection of coronary atherosclerosis and vascular imaging using intravenous contrast medium enhancement is currently available, these methods and media are dependent on many complex factors, including the type of media, volume, concentration, injection technique, catheter size and site, imaging technique, cardiac output and tissue characteristics. Only some of these factors are controllable by radiologists (see, e.g., Bae, K. T., Heikin, J. P. and Brink, J. A. (1998) Radiology 207:647-655 and Bae, K. T., Heikin, J. P. and Brink, J. A. (1998) Radiology 207:657-662). For example, mixing or streak artifacts can compromise interpretation of computed tomography (CT) scans of the abdomen. These artifacts are primarily related to the first pass (arterial phase) effects of intravenous contrast on vascular enhancement (see, e.g., Silverman, P. M. et al. (1995) Radiographics 15:25-36 and Herts, B. R., Einstein, D. M. and Paushter, D. M. (1993) J. Roentgenol. 161:1185-1190). Diffusion of contrast media outside the vascular space not only degrades lesion conspicuity, but also requires that imaging be formed within two minutes after the start of injection. Very rapid elimination through the kidneys renders these substances unsuitable for imaging of the vascular system since they cannot provide acceptable contrasts for a sufficient time. All of these difficulties are accentuated in indications that require a consistent contrast enhancement of the vascular blood pool in various vascular beds. Accordingly, improved imaging methods and contrast agents addressing these limitations will have broad clinical utility.

### SUMMARY OF THE INVENTION

The present invention provides, at least in part, compositions and methods for imaging the perfusion and extravasation of blood out of vascular tissue, including but not limited to, vascular beds (e.g., arterial and venous beds), organ tissues (e.g., myocardial tissues and other organ tissues), and tumors. The present invention is also directed to compositions and methods for imaging, detecting, or evaluating accumulated macrophages, e.g., activated macrophages, and vascular plaque, e.g., vulnerable plaque. Accordingly, in one aspect, the invention provides methods for detecting or evaluating accumulated macrophages in a blood vessel of a subject comprising administering to the subject an effective amount of a nanoparticulate contrast agent and detecting the agent thereby forming an image of said accumulated macrophages in the vessel. In another aspect, the invention provides methods for detecting or evaluating plaque, e.g., vulnerable plaque, accumulation in a vessel of a subject comprising administering to the subject an effective amount of a nanoparticulate contrast agent and detecting said agent thereby forming an image of said accumulated plaque in said vessel.

In a further aspect, the invention provides methods for predicting risk of vascular disease or disorder by detecting or evaluating accumulated macrophages within a blood vessel of a subject comprising administering to the subject an effective amount of a nanoparticulate contrast agent, detecting said agent thereby forming an image of said accumulated macrophages in the vessel, and predicting risk of vascular disease in the subject based on the accumulation of contrast agent in the vessel of the subject. In one embodiment, the vascular disease is selected from the group consisting of atherosclerosis, coronary artery disease (CAD), myocardial infarction (MI), ischemia, stroke, peripheral vascular diseases, and venous thromboembolism. In another embodiment, the methods of predicting risk of a vascular disease or disorder may be used in combination with other known risk factors for vascular diseases or disorders.

In a further aspect, the invention provides methods for detecting or evaluating the perfusion status of an organ, e.g., kidney, liver, lung, spleen, brain, heart, or pancreas in a subject, comprising administering to the subject an effective amount of a nanoparticulate contrast agent and detecting the contrast agent, thereby forming an image of said organ. In one embodiment, the method includes evaluating said image to determine said perfusion status of the organ.

In yet a further aspect, the invention provides methods for detecting or evaluating the microperfusion status of a small blood vessel, e.g., a capillary, comprising administering to the subject an effective amount of a nanoparticulate contrast agent, and detecting the agent to, thereby forming an image of the microperfusion status of the small blood vessel. In one embodiment, the method includes evaluating the image to determine said microperfusion status of said vessel. In a related aspect, the invention provides methods for detecting or evaluating the perfusion status of a tumor in a subject comprising administering to the subject an effective amount of a nanoparticulate contrast agent, and detecting the agent thereby forming an image of said perfusion status of said tumor.

In a further aspect, the invention provides methods for monitoring treatment of a tumor in a subject comprising administering to the subject an effective amount of a nanoparticulate contrast agent, and detecting said agent thereby forming an image of said perfusion status of said tumor, wherein a decrease in perfusion of the tumor compared to perfusion of the tumor prior to treatment indicates effective treatment of said tumor.

In a yet another aspect, the invention provides methods for assessing organ damage in a subject comprising administering to the subject an effective amount of a nanoparticulate contrast agent, detecting the agent thereby forming an image of the organ, and determining organ damage based on the image.

In still another aspect, the invention provides methods for assessing leakage of blood from vessels in a subject comprising administering to said subject an effective amount of a nanoparticulate contrast agent, detecting said agent thereby forming an image of the blood vessel and the area surrounding the blood vessel, and determining leakage of the blood vessel based on the image.

In one embodiment of the invention, the nanoparticulate contrast agent is a non-water soluble contrast agent. In another embodiment, the nanoparticulate contrast agent comprises a heavy element iodine or barium. In a preferred embodiment, the contrast agent is PH-50.

In a further embodiment, the mean size of the particles comprising the nanoparticulate contrast agent is about 20 nanometers to about 750 nanometers. In a preferred embodiment, the mean size of the particles comprising the nanoparticulate contrast agent is preferably about 200 nanometers to about 400 nanometers, more preferably less than about 300 nanometers.

In still another embodiment, imaging is carried out by x-ray imaging, ultrasonagraphy, computed tomography (CT), computed tomography angiography (CTA), e.g., coronary angiography, or angiography in other vascular areas (e.g., kidney, brain, liver, etc.), electron beam (EBT), magnetic resonance imaging (MRI), magnetic resonance angiography (MRA), or positron emission tomography.

In yet another embodiment, detection of the agent occurs greater than about 10 minutes after administration, preferably about 15 minutes after administration, and more preferably about 30 minutes, or more, after administration.

In a further aspect, the invention provides compositions comprising a non-water soluble, nanoparticulate with a mean particle size to allow the nanoparticulate to be taken up by activated macrophages. In one embodiment, the nanoparticulate is labelable with a contrast agent, e.g., iodine.

### BRIEF DESCRIPTION OF THE DRAWINGS

FIG. 1 depicts the chemical structure of PH-50.

FIG. 2 depicts uptake of PH-50 in the small arterioles of the lung of a rabbit.

FIG. 3 depicts uptake of PH-50 in the small arterioles of the spleen of a rabbit.

FIG. 4 depicts uptake of PH-50 in the small arterioles of the liver of a rabbit.

### DETAILED DESCRIPTION OF THE INVENTION

The present invention provides, at least in part, compositions and methods for imaging, detecting, and evaluating imaging cavities and blood pools as well as the perfusion and extravasation of blood out of vascular tissue, including but not limited to, vascular beds (e.g., arterial and venous beds), organ tissues (e.g., myocardial tissues and other organ tissues), and tumors, e.g., for the measurement of angiogenesis or perfusion status of tumors. The imaging of vascular beds in the coronary arteries and other vascular areas is important for the prediction and/or diagnosis of localized and generalized diseases and disorders and/or organ, tissue, or vessel damage (e.g., ischemic, inflamed, injured, infected, or healing organs, tissues, or vessels, vascular wall damage, peripheral vascular disease, and the like). The present invention also provides for imaging micro-perfusion of organ tissues and tumors at the level of the smallest blood vessels (i.e., capillaries). The invention is not limited to the particular vascular tissue, vascular beds or organ tissues imaged.

One aspect of the invention features a method for detecting or evaluating the perfusion status of an organ or a tumor comprising administering to a subject an effective amount of a nanoparticulate contrast agent and detecting the agent.

The present invention is also directed to compositions and methods for imaging, detecting, and evaluating macrophages, e.g., activated macrophages, and vascular plaque, e.g., vulnerable plaque. Vulnerable plaques contain macrophages, e.g., activated macrophages, which accumulate on arterial walls. The contrast agents of the invention are taken up by macrophages, e.g., activated macrophages. Therefore, visualization of the plaque containing the macrophages is possible using routine imaging technology, e.g., by x-ray imaging, ultrasonagraphy, computed tomography (CT), computed tomography angiography (CTA), electron beam (EBT), magnetic resonance imaging (MRI), magnetic resonance angiography (MRA), positron emission tomography, and other imaging technologies.

When administered to a subject, the preferred contrast agents of the invention, e.g., PH-50, remain substantially confirmed to the intravascular space and therefore do not permeate to the interstitial space or extrastitial fluids, thus facilitating the imaging of blood pools and vascular structures, e.g., vascular tissue, vascular beds, and organ tissues, as well as plaque, e.g., vulnerable plaque and macrophages. Furthermore, preferred contrast agents of the invention, e.g., PH-50, are excreted from the body via the hepatic system rather than the renal system, and therefore remain in the body for a longer period of time than agents which are excreted via the renal system. Furthermore, excretion via the hepatic system permits usage of the preferred contrast agents of the invention, e.g., PH-50, in patients with renal insufficiency, and also permits imaging of the renal system including the abdominal aorta and renal arteries to diagnose pathological conditions of the renal system, e.g., hypertension, diabetes, or cancer, e.g., kidney tumors. Furthermore, it is believed that such contrast agents, e.g., PH-50, will not cause renal system damage.

Certain embodiments of the invention feature contrast agents, e.g., PH-50, which remain in the vascular structures for an extended period of time at functionally active concentrations with a half-life of about 30-60 minutes until the contrast agent is metabolized by the liver. As such, multiple images may be taken after a single, low-dose administration of the contrast agent. Furthermore, this functional half-life time is long enough to allow vascular scanning in vascular beds of interest (kidney, liver, heart, brain and elsewhere), to be performed. This is in contrast to agents currently in use which diffuse quickly, e.g., after several seconds or minutes, allowing only a small window of time to perform imaging following administration of the agent. Furthermore, because the preferred contrast agents of the invention are substantially confined to the vascular space, whole body vascular imaging, as well as imaging of whole body plaque burden, is allowed using routine imaging technology known to those of skill in the art, e.g., x-ray to imaging, ultrasonagraphy, computed tomography (CT), computed tomography angiography (CTA), electron beam (EBT), magnetic resonance imaging (MRI), magnetic resonance angiography (MRA), and positron emission tomography. In addition, the minimal diffusion of the contrast agents of the invention from the intravascular space allows imaging of areas of vascular disease or disorder, or vascular damage, e.g., leakage, tissue damage, or tumors, to be visualized due to the accumulation of the contrast agent in areas outside of the intravascular space. The present invention also provides methods and compositions for prediction and/or diagnosis of thrombotic or thromboembolic event.

The terms “vasculature,” “vessels,” and “circulatory system” are intended to include all vessels through which blood circulates, including, but not limited to veins, arteries, arterioles, venules and capillaries. The blood vascular system is commonly divided into the macrovasculature (e.g., vessels having a diameter of >0.1 mm) and microvasculature (e.g., vessels having a diameter <0.1 mm). As used herein, the term “capillary” includes any one of the minute vessels that connect the arterioles (e.g., the smallest divisions of the arteries located between the muscular arteries and the capillaries) and venules (e.g., the minute vessels that collect blood from the capillary plexuses and join together to form veins), forming a network of nearly all parts of the body. Their walls act as semipermeable membranes for the interchange of various substances, including fluids, between the blood and tissue fluid. The average diameter of capillaries is usually between about 7 micrometers to 9 micrometers. Their length is usually about 0.25 mm to 1 mm, the later being characteristic of muscle tissue. In some instances, (e.g., the adrenal cortex, renal medulla), capillaries can be up to 50 mm long.

The term “vascular disease or disorder,” also commonly referred to as “cardiovascular disease, coronary heart disease [CHD] and coronary artery disease [CAD]” as used herein, refers to any disease or disorder effecting the vascular system, including the heart and blood vessels. A vascular disease or disorder includes any disease or disorder characterized by vascular dysfunction, including, for example, intravascular stenosis (narrowing) or occlusion (blockage) due to, for example, a build-up of plaque on the inner arterial walls, and diseases and disorders resulting therefrom. Also intended to be within the scope of the invention are thrombotic, or thromboembolic, events. The term “thrombotic or thromboembolic event” includes any disorder that involves a blockage or partial blockage of an artery or vein with a thrombosis. A thrombic or thrombolic event occurs when a clot forms and lodges within a blood vessel which may occur, for example, after a rupture of a vulnerable plaque. Examples of vascular diseases and disorders include, without limitation, atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack.

As used herein, the term “plaque,” also commonly referred to as “atheromas,” refers to the substance which builds up on the interior surface of the vessel wall resulting in the narrowing of the vessel and is the common cause of CAD. Usually, plaque comprises fibrous connective tissue, lipids (i.e. fat) and cholesterol. Frequently deposits of calcium salts and other residual material may also be present. Plaque build-up results in the erosion of the vessel wall, diminished elasticity (e.g., stretchiness) of the vessel and eventual interference with blood flow. Blood clots may also form around the plaque deposits thus further interfering with blood flow. Plaque stability is classified into two categories based on the composition of the plaque. As used herein, the term “stable” or “inactive” plaques refers to those which are calcific or fibrous and do not present a risk of disruption or fragmentation. These types of plaques may cause anginal chest pain but rarely myocardial infarction in the subject. Alternatively, the term “vulnerable” or “active” plaque refers to those comprising a lipid pools covered with a thin fibrous cap. Within the fibrous cap is a dense infiltrate of smooth muscle cells, macrophages, and lymphocytes. Vulnerable plaques may not block arteries but may be ingrained in the arterial wall, so that they are undetectable and may be asymptomatic. Furthermore, vascular plaques are considered to be at a high risk of disruption. Disruption of the vulnerable plaque is a result of intrinsic and extrinsic factors, including biochemical, haemodynamic and biomechanical stresses resulting, for example, from blood flow, as well as inflammatory responses from such cells as, for example, macrophages.

As used herein, the term “macrophage” refers to the relatively long-lived phagocytic cell of mammalian tissues, derived from blood monocytes. Macrophages are involved in all stages of immune responses. Macrophages play an important role in the phagocytosis (digestion) of foreign bodies, such as bacteria, viruses, protozoa, tumor cells, cell debris and the like, as well as the release of chemical substances, such as cytokines, growth factors and the like, that stimulate other cells of the immune system. Macrophages are also involved in antigen presentation as well as tissue repair and wound healing. There are many types of macrophages, including aveolar and peritoneal macrophages, tissue macrophages (histiocytes), Kupffer cells of the liver and osteoclasts of the bone, all of which are within the scope of the invention. Macrophages may also further differentiate within chronic inflammatory lesions to epitheliod cells or may fuse to form foreign body giant cells (e.g., granulomas) or Langerhan giant cells.

### 1. Contrast Agents of the Invention

The contrast agents of the present invention include any substance that can be introduced, e.g., injected, into a structure, e.g., an organ, tissue, blood vessel, blood pool, or plaque, and, because of the difference in the absorption of detection medium, e.g., x-rays, radiowaves, soundwaves or the like, between the contrast agent and the structure, allow for detection, visualization, or enhanced visualization, e.g., radiographic or sonographic visualization, of the structure, e.g., the organ, tissue, blood vessel, blood pool, or plaque. The contrast agents of the invention remain substantially confined to the intravascular space and therefore do not permeate to the interstitial space or extrastitial fluids. In certain embodiments, the contrast agent is of such size to allow for phagocytosis by a macrophage, e.g., an activated macrophage. Preferably, the contrast agent is a nanoparticle. In another embodiment, the contrast agents of the invention are non-water soluble. In still another embodiment, the contrast agents of the invention comprise, or are labeleable with, a heavy element, e.g., iodine or barium, which may or may not be radioactively labeled. For example, the concentration of the heavy element, e.g., iodine, may be in a 2:1 ratio of labelable compound to iodine. In still another embodiment, the contrast agents of the invention have a half-life in the vasculature of a subject of at least about 30 minutes. In yet another embodiment, the contrast agent has a neutral pH.

The compounds suitable for use in the methods of the invention include those compositions described in, for example, U.S. Pat. Nos. 5,322,679, 5,466,440, 5,518,187, 5,580,579, and 5,718,388, the contents of which are hereby incorporated by reference in their entirety.

In one embodiment, the contrast agent used in the methods of the invention is an ester of diatrizoic acid. In another embodiment, the contrast agent used in the methods of the invention is an iodinated aroyloxy ester. In still another embodiment, the contrast agent used in the methods of the invention is PH-50 (also referred to as WIN 67722 and N1177). PH-50 is an iodinated aroyloxy ester with the empirical formula C₁₉H₂₃I₃N₂O₆, and the chemical name 6-ethoxy-6-oxohexy-3,5-bis(acetylamino)-2,4,6-triiodobenzoate. PH-50 is cross-linked in a polymeric form and milled to generate nanoparticles and is non-soluble, e.g., non-water soluble.

In one embodiment, PH-50 formulated for use as a contrast agent comprises 150 mg/ml PH-50, 150 mg/ml polyethylene glycol 1450 NF, 30 mg/ml poloxamer 338. In addition, 0.36 mg/ml tromethamine, sufficient to buffer to neutral pH, is also used. In one embodiment, the pH of PH-50 may be about 7.4.

The polymeric excipients poloxamer 338 and polyethylene glycol 1450, serve as particle stabilizers and are also intended to retard the rate of plasma clearance of particles by the reticuloendothelial system (RES) after intravascular administration. Poloxamer 338 is purified by diafiltration as a part of the manufacturing process to reduce the level of low-molecular weight polymer. Other appropriate excipients or particle stabilizers may also be used.

The physicochemical properties of the contrast agents of the invention are such that one would expect slow dissolution from a subcutaneous injection site providing for slow systemic absorption and metabolic attack by plasma and/or tissue esterases once the solubilized drug is absorbed. Additionally, some of the particles are transported in the lymphatics to regional lymph nodes. Macrophage engulfment of particles and subsequent phagocytosis can also occur at the injection site and within the regional lymph nodes.

In addition, in one embodiment, intravenous administration of the contrast agent of the invention, e.g., PH-50, results in uptake by macrophages in the RES, e.g., liver, spleen, or bone marrow, with subsequent intracellular dissolution and/or metabolism, and/or redistribution into plasma.

The term “nanoparticulate” or “nanoparticle” refers to a composition comprising particles having a mean diameter of preferably between about 20.0 nanometers and about 2.0 microns, typically between about 100 nanometers and 1.0 micron. In a preferred embodiment, the nanoparticulate contrast agent used in the methods of the invention has a mean particle size of about 20 nanometers to about 750 nanometers. In another preferred embodiment, the nanoparticulate contrast agent has a mean particle size of about 200 nanometers to about 400 nanometers, even more preferably about 300 nanometers to about 350 nanometers. In a particularly preferred embodiment, the nanoparticulate contrast agents have a mean particle size of less than about 300 nanometers. A nanoparticulate composition comprises a range of particle sizes. A “mean” particle size refers to the mean radius of the particles within a composition comprising a distribution of particle sizes. Particles smaller and larger than the mean size are also included in the invention. In another preferred embodiment, the nanoparticulate contrast agent is milled to achieve a particle size distribution of 50% not more than 350 nanometers and 90% not more than 1,200 nanometers.

The term “nanoparticulate contrast agent” includes any substance that can be introduced, e.g., injected, into a structure, e.g., an organ, tissue, blood vessel, blood pool, or plaque, and, because of the difference in the absorption of detection medium, e.g., x-rays, radiowaves, soundwaves or the like, between the contrast agent and the structure, allow for detection, visualization, or enhanced visualization, e.g., radiographic or sonographic visualization, of the structure, e.g., the organ, tissue, blood vessel, blood pool, or plaque, and which is comprised of particles having a mean diameter of preferably between about 20.0 nanometers and about 2.0 microns. Preferred nanoparticulate contrast agents are comprised of particles having a mean diameter of about 100 nanometers and 1.0 micron, about 20 to about 750 nanometers, about 200 nanometers to about 400 nanometers, or about 300 nanometers to about 350 nanometers. In a particularly preferred embodiment, the nanoparticulate contrast agent is comprised of particles having a mean diameter of less than about 300 nanometers.

It is to be understood that the mean particle size of the contrast agents used in the methods of the invention may vary depending on the desired use, e.g., mean nanoparticulate size may vary for use for blood pool imaging, microperfusion, perfusion, or plaque imaging. It is also understood that varying the size of the nanoparticulate may increase or decrease side-effects and therefore the mean particle size may be adjusted to avoid unwanted side-effects. For example, nanoparticulates comprising a smaller mean size may result in fewer side-effects in a subject.

Methods of making finely milled or divided particles of drugs and drug carriers are well known in the art and the size and size range of such particles in pharmaceutical compositions can be closely controlled. For example, the nanoparticulate contrast agents used in the methods of the invention may be produced by any process known in the art for the production of the desired particle size, or by methods described in, for example, U.S. Pat. Nos. 5,718,388 and 5,518,187.

### II. Methods of Use

A. Macrophage and Vascular Plaque Imaging

Recent evidence suggests that inflammation in the vasculature, such as the coronary arteries, may be intimately involved in the development of atherosclerosis and its associated acute coronary syndromes. As a part of this inflammatory response, macrophage cells migrate to and accumulate at the site of plaque formation. Accordingly, one aspect of the invention provides a method of detecting or evaluating accumulated macrophages in a blood vessel, e.g., an artery such as a coronary or pulmonary artery, by administering, e.g., intravenously, to a subject, e.g., a mammal, such as a human, an effective amount of a contrast agent so as to detect the agent and form an image of the accumulated macrophage in the vessel. Furthermore, the invention includes methods for detecting ischemic, inflamed, injured, or infected tissues, or vessels, vascular wall damage, and the like, using the contrast agents of the invention based on the imaging and detection of macrophages, e.g., activated macrophages, at the site of ischemia, inflammation, injury, or infection based on detection of accumulated macrophages. In another embodiment, macrophage accumulation in extravascular space may also be detected. Where the contrast agent is present in the extravascular space due to, e.g., leakage, abscess, or lesions of the vascular wall, accumulation of macrophages may be detected in areas of ischemia, inflammation, injury, or infection based on detection of accumulated macrophages. Accordingly, inflammation, or inflammatory diseases or disorders, such as, but not limited to, rheumatoid arthritis, chronic pulmonary inflammatory disease, psoriasis, rheumatoid spondylitis, ostcoarthritis and gouty arthritis, allergy, multiple sclerosis, autoimmune diabetes, autoimmune diseases or disorders, and nephrotic syndrome may be detected or diagnosed. Moreover, healing or treatment of tissues or vessels, or in the extravascular space, may also be visualized by the methods of the invention by imaging the accumulation of activated macrophages at the injured site prior to treatment and post-treatment.

Yet another aspect of the invention pertains to a method of detecting or evaluating plaque, e.g., vulnerable plaque, accumulation in a vessel, tissue, or organ of a subject by administering, e.g., intravenously, an effective amount of a contrast agent of the present invention to the subject and detecting plaque accumulation in the vessels.

The invention provides for visualization, e.g., detection or imaging, of the contrast agent using any imaging techniques which are well-known in the art. These techniques may include, but are not limited to, x-ray imaging, ultrasonagraphy, computed tomography (CT), computed tomography angiography (CTA), electron beam (EBT), magnetic resonance imaging (MRI), magnetic resonance angiography (IRA), and positron emission tomography. Preferably, the detection is by CT.

The present invention also pertains to an imaging method for predicting risk of vascular disease by detecting or evaluating accumulated macrophages within a blood vessel of a subject by administering an effective amount of the contrast agent of the present invention, detecting the agent within the subject and, based on the image obtained, predicting the risk of vascular disease in the subject. As used herein, the terms “predicting risk” and “prognosticating” refers to the assessment for a subject of a probability of developing a condition, e.g., vascular disease such as, but not limited to atherosclerosis, coronary artery disease (CAD), myocardial infarction (MI), ischemia, stroke, peripheral vascular disease, and venous thromboembolism, or a stage associated with or otherwise indicated by assessment of an image obtained from the subject administered with a contrast agent of the present invention. Recent experimental and clinical studies based on the biochemical markers of inflammation and vascular perturbation in plasma as well as in atherosclerotic tissue suggest a potential role for using biochemical markers and/or other indicators of inflammation as indicators of vascular disease (see, e.g., Van Lente, F. supra; and Schmidt, M. I. et al., supra). Accordingly, using the imaging data obtained from the methods of the present invention to image macrophages, together with other criteria such as age, obesity, cholesterol level, HDL and LDL levels, smoking, and the like which are well known to those skilled in the art, one skilled in the art will be able to predict the likelihood that the subject will develop a vascular disease or disorder or is at risk for developing a vascular disease or disorder. For example, a subject showing large macrophage accumulation together with high cholesterol and LDL levels will be at a greater risk than a subject showing little or no macrophage accumulation and low LDL levels. Imaging macrophage accumulation according to the methods of the present invention can also assist in predicting, diagnosing, or prognosticating other vascular diseases or related disorders. Such other diseases include atherosclerosis, CAD, MI, unstable angina, acute coronary syndrome, pulmonary embolism, transient ischemic attack, thrombosis (e.g., deep vein thrombosis, thrombotic occlusion and re-occlusion and peripheral vascular thrombosis), thromboembolism, e.g., venous thromboembolism, ischemia, stroke, peripheral vascular diseases, and transient ischemic attack

B. Vascular Imaging and Perfusion

The invention further provides methods for imaging cavities and blood pools, imaging of anatomy, e.g., tissues and organs, e.g., including, for example, cardiac, vascular, lung, kidney, hepatic, liver, spleen, or brain tissue, and structure of vessels, e.g., to angiography, and imaging of organ and tissues, e.g., including, for example, vascular, hepatic, cardiac, liver, spleen, or brain tissue perfusion, including microperfusion of small vessels, e.g., capillaries.

The invention can be used to image microperfusion in organ tissues to assess the perfusion status of organs on the level of the smallest blood vessels, e.g., capillaries. Tissues and organs, e.g., kidneys, liver, brain, and lung, can be monitored for adequate blood supply and blood perfusion. This ability can be used in assessing organ damage associated with angina pectoris or heart attacks, stroke, or vascular damage or injury, thereby replacing the currently utilized Technetium99 scans, or the imaging of brain perfusion to assess pathological events (stroke, tumors, and the like), to assess vessel leakages (aneurisms and diffuse bleedings after trauma or other pathological events), or to determine the microperfusion status of tumors including monitoring of treatment effects for all these applications (including the effectiveness of anti-angiogenic treatment, surgical intervention, and other treatments). Furthermore, vessels may be imaged in order to assess occlusion due to build-up of plaque and assess the necessity of surgical procedures, e.g., bypass surgery or other invasive or non-invasive treatment, e.g., lifestyle changes, including, for example, changes in diet, or medication. Imaging contrast in small blood vessels is indicative of an active perfusion of these tissue areas and allows important conclusions on the health and viability of the tissue that is being imaged.

In one embodiment, the contrast agents of the present invention can be used for angiography to diagnose, e.g., blockage of an artery, e.g., a peripheral artery, a coronary artery, or kidney arteries. Angiography can identify the exact location of the blockage and can assess the severity of the blockage, based on the image generated. Occlusions may also be detected as well as the percent of blockage of the artery. Angiography may also detect the presence of an aneurysm and may be used prior to surgery to assess the location and severity of the aneurysm.

Furthermore, the invention provides methods for imaging the perfusion status, e.g., microperfusion status, of tumors, e.g., measurement of angiogenesis in tumors. The growth of tumors to a clinically relevant size is dependent upon an adequate blood supply. This is achieved by the process of tumor stroma generation where the formation of new capillaries is a central event. Progressive recruitment of blood vessels to the tumor site and reciprocal support of tumor expansion by the resulting neovasculature are thought to result in a self-perpetuating loop helping to drive the growth of solid tumors. The development of new vasculature also allows an ‘evacuation route’ for metastatically-competent tumor cells, enabling them to depart from the primary site and colonize initially unaffected organs. Imaging of vessels, including capillaries, within or in the area of a tumor-like mass or growth provides a method to assess or diagnose whether the mass is in fact a tumor as opposed to a non-cancerous growth, e.g., a cyst, and also provides a method to determine whether a tumor is benign or malignant and if malignant, determining the degree of malignancy based on the degree of angiogenesis of the mass. Furthermore, it has been established that the microvessels of tumors are particularly “leaky,” with permeability being high compared to the microvessel of non-tumorous, healthy and intact tissues. Therefore, the contrast agents of the invention may be used to identify tumorous tissue based on visualization of the diffusion status, or “leakiness” of the contrast agent of the invention.

Measurement of angiogeneis in tumors may also be used to monitor tumor therapy, e.g., anti-vascular therapy or other cancer therapies, wherein a decrease in angiogeneis of a tumor indicates effectiveness of the tumor therapy. The method of assessing the tumor may include a single visualization of the tumor or two or more visualizations of the tumor over a period of time, e.g., during the course of therapy. Furthermore, the contrast agents of the invention may be used to assess successful surgical treatment by assessing the presence or absence of the tumor post-surgery.

In one embodiment, the contrast agents of the invention may be used to diagnose the occurrence of stroke or to determine the risk of stroke in a subject. The contrast agents of the invention may be used to pinpoint quickly the precise location of a stroke and determine the extent of damage, to assess the blood flow throughout the brain, to distinguish between an ischemic or hemorrhagic stroke, to determine the extent of damage, to determine the present of regarding collateral (alternative) blood vessels in the brain, or to diagnose blockage in the carotid arteries.

In one embodiment, the agent is administered by being injected intravenously or intra-arterially, whereupon imaging of the vascular beds or tissue areas can be achieved by using computed tomography techniques or other x-ray containing imaging techniques.

In another embodiment, several imaging procedures may be performed following a single administration of the contrast agent of the invention, e.g., PH-50. For example, assessment of the risk for or presence of vascular disease may be carried out by imaging anatomy and structure of the vessels, e.g., coronary angiography, imaging of tissue perfusion, and imaging of cavities, e.g., heart cavities, during one imaging session. Furthermore, the lack of diffusion of the contrast agents of the invention out of the vascular space also allows for whole body vascular imaging as well as imaging of whole body plaque burden, using routine imaging technology known to those of skill in the art.

### III. Imaging Technology Used in the Methods of the Invention

As used herein, the term “imaging” or “clinical imaging” refers to the use of any imaging technology to visualize a structure, e.g. a blood vessel, e.g., a capillary, blood pool, or plaque, either in vivo or ex vivo by measuring the differences in absorption of energy transmitted by or absorbed by the tissue. Imaging technology includes x-ray technology, scanning thermography such as ultrasonagraphy, computed tomography (CT), magnetic resonance (MRI or NMR), and radionucleotides, i.e., ¹²³I or ¹²⁵I, for use in techniques such as positron emission tomography and the like.

CT imaging involves measuring the radiodensity of matter. Radiodensity is typically expressed in Hounsefield Units (HU). Hounsefield Units are a measure of the relative absorption of computed tomography X-rays by matter and is directly proportional to electron density. Water has been arbitrarily assigned a value of 0 HU, air a value of −1000 HU, and dense cortical bone a value of 1000 HU. Conventional CT scanners produce a narrow beam of x rays that passes through the subject and is picked up by a row of detectors on the opposite side. The tube and detectors are positioned on opposite sides of a ring that rotates around the patient, although the tube is unable to rotate continuously. After each rotation the scanner must stop and rotate in the opposite direction. Each rotation acquires an axial image of approximately 1 cm in thickness, at approximately 1 second per rotation. The table moves the patient a set distance through the scanner. Spiral (helical) CT scanners comprise a rotating tube, which allows a shorter scan time and more closely spaced scans. Angiography is possible with spiral scanning. Multislice CT scanners are considered “supercharged” spiral scanners. Where conventional and spiral scanners use a single row of detectors to pick up the x-ray beam, multislice scanners have up to eight active rows of detectors. Multislice scanners give faster coverage of a given volume of tissue. Various types of CT technology used in clinical practice is described in, for example, Garvey, C. and Hanlon, R. (2002) BMJ 324:1077.

In CTA, iodinated contrast agents are injected intravenously and images are obtained. Highly detailed images of the vasculature are generally obtained using CTA by reformatting the axial images to yield a composite picture of the vessels. During this reformatting, the picture of the vasculature is optimized based on the measured density in the vessels being visualized. To perform this imaging, various baseline image subtractions are performed.

CT imaging techniques which are employed are conventional and are described, for example, in Computed Body Tomography, Lee, J. K. T., Sagel, S. S., and Stanley, R. J., eds., 1983, Ravens Press, New York, N.Y., especially the first two chapters thereof entitled “Physical Principles and Instrumentation”, Ter-Pogossian, M. M., and “Techniques”, Aronberg, D. J., the disclosures of which are incorporated by reference herein in their entirety.

In one embodiment, the methods of the invention are carried out by the following procedure. A series of CT images is acquired with appropriate temporal resolution beginning just prior to contrast medium administration and continuing through the period of contrast agent administration (1-30 seconds, 1 minute, 5 minutes, 10 minutes, 15 minutes, 20 minutes, 30 minutes, 40 minutes, 50 minutes, 60 minutes, 90 minutes, 120 minutes, or more) and for a selected time period after the administration. In another embodiment, imaging is carried out after administration of the contrast agent. A wide range of image acquisition periods can be used in the method of the invention.

For example, in one embodiment, the selected time period is from about 10 seconds postcontrast to about 10 hours postcontrast, from about 30 seconds postcontrast to about 3 hours postcontrast, more preferably from about 50 seconds postcontrast to about 1 hour postcontrast, or more preferably still from about 1 minute postcontrast to about 10 minutes postcontrast. In another embodiment, the selected time period is from the time of completion of the contrast agent to about 30, 40, 50, 60 seconds postcontrast, to about 5, 10, 15, 20, 30, 40, 50, 60 minutes postcontrast, or to about 1, 2, 3, 4, 5, 6, 7, 8, 9, or more hours post contrast. Multiple images or series of images may be taken after a single administration of a contrast agent of the invention, e.g., PH-50.

A typical series might include an image every five seconds before and during the contrast medium administration, slowing further to an image every ten seconds for the subsequent three minutes, and finally slowing to an image every 30 seconds until the 10 minute completion of the series. These serial images are used to generate the dynamic enhancement data from the tissue and from the blood as measured in a vessel to be used for kinetic modeling and, ultimately, to the calculation of blood volume and perfusion within the tissue of interest. After the completion of the dynamic acquisition localized to to the region-of-interest, it may be elected to acquire additional CT images of the patient in other anatomic sites to extract additional diagnostic data or for delayed images in the same site. After CT scanning, the subject is removed from the scanner unit, and the intravenous catheter used for injection of the contrast agent can be removed. The data acquired from the CT imaging procedure is processed to provide the necessary information.

The contrast enhanced CT images can be used, for example, to define the location, caliber, and flow characteristics of vascular structures within the scanned anatomic regions as well as macrophage accumulation and plaque accumulation. Moreover, the images can be utilized to monitor the effect of potentially therapeutic drugs which are expected to alter perfusion status, e.g., microvascular perfusion status.

The methods described herein are useful with substantially any tissue type. In one embodiment, the tissue is a member selected from the group consisting of normal tissue, diseased tissue, and combinations thereof. In a further preferred embodiment, the tissue is at least partially a diseased tissue and the diseased tissue is a member selected from the group consisting of tissues which are neoplastic, ischemic, hyperplastic, dysplastic, inflamed, traumatized, infarcted, necrotic, infected, healing and combinations thereof

### IV. Pharmaceutical Compositions

Another aspect of the present invention provides pharmaceutically-acceptable compositions which comprise a nanoparticulate contrast agent formulated with one or more pharmaceutically-acceptable carrier(s), in an amount effective to allow imaging of blood pools, vascular tissue perfusion and the extravasation of blood out of vessels, to detect macrophages, or to detect plaques, e.g., vulnerable plaque, within the vessels of a subject.

In a particular embodiment, the nanoparticulate contrast agent is administered to the subject using a pharmaceutically-acceptable formulation, e.g., a pharmaceutically-acceptable formulation that suitable for administration in liquid form, including parenteral administration, for example, by intravenous injection, either as a bolus or by gradual infusion over time, intraperitoneally, intramuscularly, intracavity, subcutaneously, transdermally, dermally or directed directly into the vascular tissue of interest as, for example, a sterile solution or suspension.

In certain embodiments, the subject is a mammal, e.g., a primate, e.g., a human. As used herein, the language “subject” is intended to include human and non-human animals. Preferred human animals include a human patient suffering from, or prone to suffer from, a vascular disease, thrombotic disease, stroke, or cancer, e.g., tumors. The term “non-human animals” of the invention includes all vertebrates, e.g., mammals, e.g., rodents, e.g., mice, and non-mammals, such as non-human primates, sheep, dogs, cows, chickens, rabbits, amphibians, reptiles and the like.

The phrase “pharmaceutically acceptable” is employed herein to refer to those nanoparticulate contrast agents of the present invention, compositions containing such contrast-agents, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as poloxamer 338 and polyethylene glycol 1450; (10) oils, such as peanut oil, cottonseed oil, safflower oil, sesame oil, olive oil, corn oil, and soybean oil; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Methods of preparing these compositions include the step of bringing into association a nanoparticulate contrast agent with the carrier and, optionally, one or more accessory ingredients. Usually, the formulations are prepared by uniformly and intimately bringing into association a contrast agent with liquid carriers.

Liquid dosage forms for oral administration of the contrast agent(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active nanoparticulate contrast agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more contrast agent(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Pharmaceutical compositions of this invention suitable for parenteral administration comprise one or more contrast agent(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, e g, F108.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of the contrast agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of nanoparticulate contrast agent(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the nanoparticulate contrast agent(s) is administered as a pharmaceutical, to humans and animals, it can be given per se or as a pharmaceutical composition containing, for example, 0.1 to 99.5% (more preferably, 0.5 to 90%) of active ingredient in combination with a pharmaceutically-acceptable carrier.

The term “administration” or “administering” is intended to include routes of introducing the nanoparticulate contrast agent(s) to a subject to perform their intended function. Examples of routes of administration which can be used include, for example, injection (subcutaneous, intravenous, parenterally, intraperitoneally, intrathecal. The pharmaceutical preparations are, of course, given by forms suitable for each administration route. For example, these preparations are administered, for example, by injection. The injection can be bolus or can be continuous infusion. Depending on the route of administration, the nanoparticulate contrast agent can be coated with or disposed in a selected material to protect it from natural conditions which may detrimentally effect its ability to perform its intended function. The nanoparticulate contrast agent can be administered alone, or in conjunction with either another agent as described above or with a pharmaceutically-acceptable carrier, or both. The nanoparticulate contrast agent can be administered prior to the administration of the other agent, simultaneously with the agent, or after the administration of the agent. Furthermore, the nanoparticulate contrast agent can also be administered in a proform which is converted into its active metabolite, or more active metabolite in vivo.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically”, “peripheral administration” and “administered peripherally” as used herein mean the administration of a nanoparticulate contrast agent(s), drug or other material, such that it enters the patient's system and, thus, is subject to metabolism and other like processes, for example, subcutaneous administration.

Regardless of the route of administration selected, the nanoparticulate contrast agent(s), which may be used in a suitable hydrated form, and/or the pharmaceutical compositions of the present invention, are formulated into pharmaceutically-acceptable dosage forms by conventional methods known to those of skill in the art.

To use the nanoparticulate contrast agents of the present invention, the contrast agent is given in a dose which is diagnostically effective. A “diagnostically effective amount” or “effective amount” of a nanoparticulate contrast agent of the present invention is typically an amount such that when administered in a physiologically tolerable composition is sufficient to enable detection of vascular sites, macrophage accumulation, and/or plaque, e.g., vulnerable plaque, within the subject. Typical dosages can be administered based on body weight, and typically are in the range of about 0.1 mL/kg to about 8.0 mL/kg, about 0.2 mL/kg to about 7.0 mL/kg, about 0.3 mL/kg to about 6.0 mL/kg, about 4 mL/kg to about 5.5 mL/kg, about 0.5 mL/kg to about 4.0 mL/kg, about 0.6 mL/kg to about 3.5 mL/kg, about 0.7 mL/kg to about 3.0 mL/kg, about 0.8 mL/kg to about 2.5 mL/kg, about 0.9 mL/kg to about 2.0 mL/kg, or about 1.0 mL/kg to about 1.5 mL/kg, based on a stock solution of about 150 mg/mL consisting of about 15% weight/volume [w/v]. The administration of the contrast agent of the invention may be over a period of time, e.g., by infusion, or by a single administration. In one embodiment, the administration rate of the contrast agent is about 0.6 mL/sec to about 3 mL/sec.

The dosage of the nanoparticulate contrast may also vary with the radioactivity of a radioisotope and will be taken into account in determining a suitable dose to be given of the contrast agent of the present invention. For example, the mean lethal dosages of both ¹²⁵I and ¹²³I have been calculated at about 79+/−9 cGy (in Chinese hamster ovary cells; see, e.g., Makrigiorgos, et al Radiat Res. 11:532-544). For diagnostic purposes, the dosage will be less than the mean lethal dose for the radioisotope.

For example, with respect to the half-life of common radioisotopes, the half-life of ¹²³I at a dose between 1 and 20 mCi is about 13 hours, while the half-life of ¹³¹I at a dose of less than 5 mC is about 8 days. It is expected that a useful dose of ¹²³I-labeled contrast agent would be between 1 and 20 mCi, while less than 5 mCi of the longer-lived ¹³¹I would be used (e.g. 0.5-5 mCi). Thus, for use according to the present invention, the preferred dose of agents including radioisotopes with longer half-lives will be less than the preferred dose of agents including radioisotopes with shorter half-lives.

Compositions comprising the nanoparticulate contrast agent are conventionally administered intravenously, as by injection of a unit dose, for example. The term “unit dose” when used in reference to the nanoparticulate contrast agent of the present invention refers to physically discrete units suitable as unitary dosage for the subject, each unit containing a predetermined quantity of active material calculated to produce the desired effect in association with the required diluent, i.e., carrier, or vehicle. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a desired effect. Generally, out of one hundred percent, this amount will range from about 1 percent to about ninety-nine percent of active ingredient, preferably from about 5 percent to about 70 percent, most preferably from about 10 percent to about 30 percent.

The nanoparticulate contrast agent is administered in a manner compatible with the dosage formulation, and in an effective amount. The quantity to be administered depends on the subject, capacity of the subject's system to utilize the active ingredient, the degree of contrast desired, and the structure to be imaged. Precise amounts of the contrast agent required to be administered depend on the judgement of the practitioner and are peculiar to each individual. However, suitable dosage ranges for systemic application are disclosed herein and depend on the route of administration. Suitable regimes for initial administration and subsequent administration, e.g., after initial imaging, are also contemplated and are typified by an initial administration followed by repeated doses at one or more hour intervals by a subsequent injection or other administration. Bolus administration, multiple dosages or continuous intravenous infusion sufficient to maintain concentrations in the blood in the ranges for specific in vivo imaging are also contemplated. Infusion of the contrast agent may be for less than one minute, two minutes, three minutes, four minutes, five minutes, or more.

### Kits

It is anticipated that the methods and the contrast agents of the invention can be incorporated into a commercial kit or system for imaging, detecting, and evaluating the perfusion and extravasation of blood out of vascular tissue, including but not limited to, vascular beds (e.g., arterial and venous beds), organ tissues (e.g., myocardial tissues and other organ tissues), and tumors, e.g., for the measurement of angiogenesis or perfusion status of tumors, or for the imaging, detecting, and evaluating macrophage accumulation or plaque accumulation. Moreover, the method and contrast agents of the invention can be incorporated into a kit for determining the changes in tissue perfusion or microperfusion, angiogenesis, extravasation of blood, macrophage accumulation; or plaque accumulation, in response to treatment measures. The kit may contain a nanoparticulate contrast agent, and instructions for use and may further contain directions on the administration and use of the nanoparticulate contrast agent in conjunction with the appropriate imaging technology and dosage requirement for the intended use.

Other features, advantages and embodiments of the invention will be apparent from the following examples which are meant to illustrative, and therefore, not limiting in any way.

### EXAMPLES

### Example 1

### Example of Contrast Agent Used in the Methods of the Invention

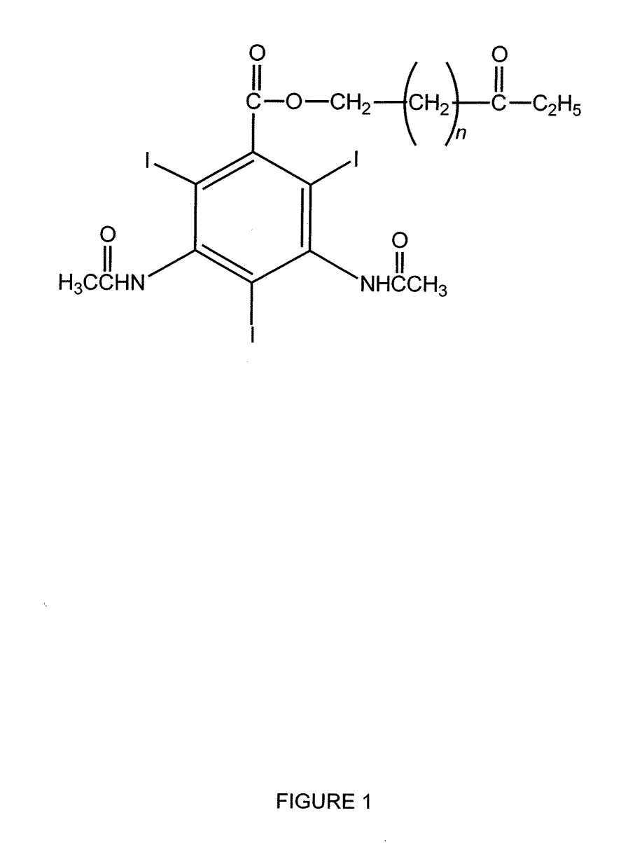

Sterile WIN 67722 Suspension 150 mg/mL (referred to herein as “Sterile PH-50”, “PH-50 Injectable Suspension” or “PH-50 drug product”) is a parenteral iodinated x-ray contrast agent which has been utilized for indirect lymphography. The PH-50 compound is described, for example, in U.S. Pat. Nos. 5,322,679, 5,466,440, 5,518,187, 5,580,579, and 5,718,388. PH-50 has the empirical formula C₁₉H₂₃I₃N₂O₆ and has the chemical name 6-ethoxy-6-oxohexy-3,5-bis(acetylamino)-2,4,6-triiodobenzoate, an esterified derivative of the x ray contrast agent diatriazoic acid. PH-50 has a molecular weight of 756.1. The structural formula for PH-50 is shown in FIG. 1. The PH50 compound can be produced by the condensation of ethyl 6-bromohexanoate with sodium diatrizoate in DMF followed by the precipitation of the product from DMSO and washing with ethanol. PH-50 can be obtained from Sigma-Aldrich Fine Chemicals.

The concentration of iodine in PH-50 Injectable Suspension is 76 mg/mL. PH-50 Injectable Suspension is a white to off-white crystalline material containing 50.35% iodine (by weight), and has a low water solubility (<10 μg/mL).

The PH-50 drug product is milled to achieve a particle size distribution of 50% not more than about 350 nanometers and 90% not more than about 1,200 nanometers. The milled dug product can be obtained from Nanosystems, Inc.

The final formulation of PH-50 Injectable Suspension is as set forth in Table 1, below:

TABLE 1
PH 50 Formulation

| | MW | Molar Conc. | Mass Conc. |
|---|---|---|---|
| Component | (g/mole) | (M) | (mg/ml) |
| PH-50 | 756.12 | 0.198 | 150 |
| Polyethylene glycol 1450 NF | ~15,000 | 0.01 | 150 |
| Poloxamer 338 | ~14,760 | 0.002 | 30 |
| Tromethamine-sufficient | 121.14 | 2.97 | 0.36 |
| to buffer to neutral pH | | | |

Relevant Formulation Specifications:
pH ~7.4
Particle Size 50% NMT 350 nm
90% NMT 1200 nm

The polymeric excipients, poloxamer 338 and polyethylene glycol 1450, serve as particle stabilizers and are also intended to retard the rate of plasma clearance of particles by the reticuloendothelial system (RES) after intravascular administration. Poloxamer 338 is purchased from BASF®, and is purified by diafiltration as a part of the manufacturing process to reduce the level of low-molecular weight polymer.

The physicochemical properties of the drug particles are such that one would expect slow dissolution from a subcutaneous injection site providing for slow systemic absorption and metabolic attack by plasma and/or tissue esterases once the solubilized drug is absorbed. Additionally, some of the particles are transported in the lymphatics to regional lymph nodes. Macrophage engulfment of particles and subsequent phagocytosis can also occur at the injection site and within the regional lymph nodes.

In addition, IV administration of PH-50 should result in uptake by macrophages in the RES, e.g., liver, spleen, bone marrow) with subsequent intracellular dissolution and/or metabolism or redistribution into plasma.

### Example 2

### Evaluation of PH-50 Nanoparticulate Contrast Material as a Vascular Imaging Agent

The primary focus of this study was to investigate the initial uptake and vascular distribution of an iodinated nanoparticle in normal New Zealand white rabbits using computed tomography (CT).

### A. Materials and Methods:

1. Test Material and Animals

The test material used for this study comprised PH-50 at 15% (w/v) (identification no.: GLP-N1177-20000005-A).

Animals used in this study were four (4) male New Zealand white rabbits obtained from NCSU-CVM. At the time of initiation, the rabbits were adult in age and weighed approximately 3.00±0.16 kg. The animals were housed separately in cages and fed a standard dry laboratory diet. Filtered tap water was provided ad labium. The animals were acclimated for fourteen (14) days prior to the start of the study. The New Zealand white rabbits were chosen because they are considered an acceptable animal model for studies of this type. The number of animals assigned to this study represented the minimum number of animals required to meet the objectives of this study.

2. Study Design and Procedures

The study design comprised assessing the initial uptake and vascular distribution of the iodinated nanoparticle, PH-50, having a mean diameter of 282 nm and a concentration of 15% (w/v) in intact male rabbits with an average weight of 3.00±0.16 kg. One (1) mL of PH-50 injected as a bolus intravenously (IV) via the jugular vein (n=2); and three (3) mL with a five (5) minute infusion via IV by way of the jugular vein (n=2); using the criteria set forth below in Table 1:

TABLE 2
Study Design

| | | | | | mg of | mg/kg | |
|---|---|---|---|---|---|---|---|
| Group | N | Treatment | Route | mL/rabbit | PH-50/rabbit | of PH-50* | CT scans |
| 1 | 1 | PH-50 | IV | 1 mL | 150 mg | 50 mg/kg | Pre-dose, |
| | | | | | | | approximately |
| | | | | | | | 30 sec, 5 and |
| | | | | | | | 15 min post, 1 |
| | | | | | | | hr and 8 hrs |
| | | | | | | | post-dose |
| 2 | 1 | PH-50 | IV | 3 mL/5 min | 450 mg | 150 mg/kg | Pre-dose, |
| | | | | | | | approximately |
| | | | | | | | 30 sec, 5 and |
| | | | | | | | 15 min post, 1 |
| | | | | | | | hr and 8 hrs |
| | | | | | | | post-dose |
| 3 | 1 | PH-50 | IV | 1 mL | 150 mg | 50 mg/kg | Digital |
| | | | | | | | angiography, |
| | | | | | | | 5 and 15 min, |
| | | | | | | | 1 hr and 8 hrs |
| | | | | | | | post dose |
| 4 | 1 | PH-50 | IV | 3 mL/5 min | 450 mg | 150 mg/kg | Digital |
| | | | | | | | angiography, |
| | | | | | | | 5 and 15 min, |
| | | | | | | | 1 hr and 8 hrs |
| | | | | | | | post-dose |

*Calculation based on a 3 kg rabbit

The animals were anesthetized via mask using Isoflorene® prior to each study and maintained under anesthesia during CT scan procedures. The animals were recovered and were mobile and allowed to drink up to the 8 hour study. Digital Subtraction Angiography (DSA) was performed during initial injection (one IV bolus and one infusion), then each rabbit was immediately moved to CT. The CT was performed after the initial injection (one IV bolus and one infusion).

All CT imaging was performed using a GE® Sytec Sri Helical Scanner (Milwaukee, Wis.) and the images were stored as DICOM3 on GE® optical and CD-Rom medium. The CT imaging parameters for these studies were 1 mm consecutive slices at 120 kvp at 1.5 sec, 200 mA. All scans were done with a standard phantom placed beneath the animal. All CT scans performed were of the entire animal (rostral nasal area to pelvis). Contrast attenuation was measured in Hounsfield units (HU) and expressed as percent (%) uptake, taking into account the calibration from the phantom. The iodine concentration and iodine uptake were estimated post-injection from vascular, lung and liver tissues.

### B. Results

The results of these experiments are summarized below in Tables 2 and 3. The results shown in Table 2 are in actual Hounsfield units for both bolus injection and infusion.

TABLE 3
Summary of Results in Hounsfield Units
for Bolus Injections and Infusion

| | | | | | | |
|---|---|---|---|---|---|---|
| Time of | | | | | | |
| Measurement post | | Posterior | Portal | | | |
| injection | Aorta | Vena Cava | Vein | Cardiac | Liver | Brain |

Hounsfield Units (HU) - Bolus Injection

| Rabbit No. 1159 | | | | | | |
|---|---|---|---|---|---|---|
| Pre-treatment | n/a | n/a | n/a | n/a | n/a | n/a |
| Pre-treatment | n/a | n/a | n/a | n/a | n/a | n/a |
| 15 min | 43.80 | 52.17 | 53.54 | 30.13 | 87.08 | 32.30 |
| 60 min | 43.83 | 65.48 | 89.92 | 44.96 | 94.17 | 34.39 |
| 480 min | 21.00 | 25.36 | 39.15 | 33.73 | 76.24 | 31.00 |
| Rabbit No. 1156 | | | | | | |
| Pre-treatment | 15.60 | 19.00 | 42.00 | 39.27 | 75.43 | 31.60 |
| 0 min | 65.00 | 51.00 | 52.00 | 54.00 | 85.00 | 29.00 |
| 5 min | 34.00 | 67.00 | 58.08 | 45.00 | 95.32 | 27.00 |
| 15 min | 40.85 | 40.31 | 79.65 | 44.02 | 91.73 | 29.03 |
| 60 min | 25.38 | 25.31 | 87.37 | 46.52 | 89.56 | 30.57 |
| 480 min | 19.20 | 36.24 | 39.73 | 40.65 | 66.15 | 30.50 |

Hounsfield Units (HU) - Infusion

| | | | | | | |
|---|---|---|---|---|---|---|
| Rabbit No. 1166 | | | | | | |
| Pre-treatment | n/a | n/a | n/a | n/a | n/a | n/a |
| Pre-treatment | n/a | n/a | n/a | n/a | n/a | n/a |
| 15 min | 20.82 | 43.09 | 63.85 | 47.51 | 117.99 | 27.15 |
| 60 min | 23.08 | 25.08 | 59.87 | 37.66 | 108.39 | 34.85 |
| 480 min | 14.30 | 39.35 | 42.92 | 35.31 | 63.20 | 38.90 |
| Rabbit No. 1151 | | | | | | |
| Pre-treatment | 34.15 | 47.46 | 36.43 | 36.34 | 86.00 | 44.06 |
| 0 min | 49.08 | 73.00 | 54.03 | 48.32 | 93.08 | 44.89 |
| 5 min | 38.85 | 68.54 | 69.54 | 58.67 | 113.68 | 51.38 |
| 15 min | 46.77 | 47.46 | 56.85 | 37.81 | 111.22 | 46.75 |
| 60 min | 47.62 | 27.00 | 34.46 | 38.49 | 104.26 | 50.38 |
| 480 min | 13.00 | 37.72 | 38.81 | 36.35 | 69.57 | 34.37 |

Table 3 shows the results of the percent uptake calculated for the two animals having pre-contrast CT scans. The percent uptake was calculated using the pre-injection node as baseline (i.e., no contrast).

TABLE 4
Summary of Results for Percent Uptake
Percent Uptake (%)

| Time of | | Posterior | Portal | | | |
|---|---|---|---|---|---|---|
| measurement | Aorta | Vena Cava | Vein | Cardiac | Liver | Brain |
| Rabbit No. 1156 | | | | | | |
| Pre-treatment | 0 | 0 | 0 | 0 | 0 | 0 |
| 0 min | 317.0 | 168.0 | 24 | 38 | 13 | −8 |
| 5 min | 118 | 253 | 38 | 15 | 26 | −15 |
| 15 min | 162 | 112 | 90 | 12 | 22 | −8 |
| 60 min | 63 | 33 | 108 | 18 | 19 | −3 |
| 480 min | 23 | 91 | −5 | 4 | −12 | −3 |
| Rabbit No. 1151 | | | | | | |
| Pre-treatment | 0 | 0 | 0 | 0 | 0 | 0 |
| 0 min | 44 | 54 | 48 | 33 | 8 | 2 |
| 5 min | 14 | 44 | 91 | 61 | 32 | 17 |
| 15 min | 37 | 0 | 56 | 4 | 29 | 6 |
| 60 min | 39 | −43 | −5 | 6 | 21 | 14 |
| 480 min | −62 | −21 | 7 | 0 | −19 | 90.1 |

** Percent uptake = Post-injection Hounsfield value (t = X hours)/Pre-injection Hounsfield value (t = 0) and expressed as a percentage.

These data demonstrate that the optimal time for imaging appears to be within the first five to fifteen minutes post injection. Due to the infusion rate and the additional volume given during injection, the infusion methods created the greatest contrast enhancement. This methods was superior for both vascular enhancement and liver visualization.

### C. Conclusions

The results of these experiments demonstrate that the iodinated nanoparticle can be used effectively and efficiently for CT vascular imaging within the first 15 minutes post-injection. The data showed a dramatic increase in Hounsfield units in the liver post-injection, thus demonstrating that PH-50 could be a useful contrasting agent for the liver. It is also important to note that no adverse reactions to the compound were noted.

### Example 2

### Evaluation of PH-50 Nanoparticulate Contrast Material in Detecting

Atherosclerotic plaques in Rabbits

The purpose of this example is to show the uptake of an iodinated nanoparticle (PH-50) by atherosclerotic plaques in New Zealand white rabbits using Computed Tomography (CT).

### A. Materials and Methods:

1. Test Material and Animals

The test material consisted of PH-50 at 15% (w/v) (identification no.: GLP-N1177-20000005-A) which is available from the laboratory or Hoyle Consulting, Inc. The handling of the test material consisted of gently inverting the vial approximately 10 times in succession prior to dosing in order to assure that the formulation was fully dispersed and mixed well. Vigorous shaking was avoided to reduce the amount of “foaming.”

Animals used in this study were six (6) male New Zealand white rabbits obtained from UCD. At the time of initiation, the rabbits were adult in age and weighed approximately 2.69±0.06 kg. The animals were housed separately in cages and fed a cholesterol enriched diet (CED) (TestDiet®—Richmond, Ind.) dry laboratory diet. Filtered tap water was provided ad labium. The animals were acclimated for a minimum of 7 days prior to the start of the study.

2. Study Design and Procedures

The study design comprised putting the rabbits on a CED for 5 weeks prior to the carotid overstretch procedure. At week 5, the carotid overstretch procedure was performed along with anticoagulant therapy (described below). The rabbits were then placed back on the CED with the anticoagulant therapy for an additional two (2) weeks. The rabbits were then anesthetized and Computer Tomography (CT) studies were performed. An iodinated nanoparticle, PH-50, having a mean diameter of 282 nm and a concentration of 15% (w/v) was evaluated in intact male rabbits with an average weight of 2.69±0.06 kg. Six (6) mL/kg of PH-50 was infused over ninety (90) minutes intravenously (IV) via an ear vein. The rabbits were then euthanized and necropsied after the 8 hour CT study. Table 4 below shows treatment given to the control and experimental animal groups.

TABLE 5
Study Design

| | | | | | mg of PH-50 | Mg/kg of | |
|---|---|---|---|---|---|---|---|
| Group A | No. | Treatment | Route | mL/rabbit | per rabbit | PH-50** | CT Scans |
| 1 | 2 | Control | IV | 1 mL of | 0 | 9 | None |
| | | (0.9% | | 0.9% | | | |
| | | sterile | | sterile | | | |
| | | saline) | | saline | | | |
| 2 | 4 | PH-50 | IV | 18 mL/ | 2700 mg | 900 | Pre-dose, |
| | | | | 90 min | | mg/kg | approximately |
| | | | | | | | 30 seconds |
| | | | | | | | post-dose, 5 |
| | | | | | | | and 15 minutes |
| | | | | | | | post-dose, 1 |
| | | | | | | | hour, 4 hour |
| | | | | | | | and 8 hours |
| | | | | | | | post-dose |

**Assuming a 3 kg rabbit

The CED diet consisted of Purina® 5321 (a standard high-quality rabbit chow that is widely available) supplemented with 2% cholesterol, 5% coconut oil and 0.5% sodium cholate. The CED was formulated by and purchased from TestDiet® (Richmond, Ind.).

The carotid overstretch procedure was performed as follows: Rabbits were pretreated with an appropriate anticoagulant (heparin) prior to the procedure, and maintained on aspirin (20 mg/kg) for 14 days after the procedure. On the day of the procedure, the rabbits were boxed down and ventilated appropriately. The left carotid artery was exposed using standard techniques known in the art. A small puncture was made distal to the site to be overstretched, and a balloon catheter was placed in the artery. The vessel diameter was estimated from direct visualization. The balloon overstretch injury was performed using balloon diameters 30% greater than the baseline arterial diameter at the target areas three inflations of the balloon at approximately 6 atmospheric pressure for 30 seconds each, with a one-minute interval between inflations. Afterwards, the catheter was withdrawn and the rabbits allowed to recover for 14 days prior to the administration of PH-50. The animals were maintained on the CED and aspirin therapy during the 14-day recovery period. Fourteen (14) days following the overstretch procedure, the rabbits were anesthetized and maintained under anesthesia during PH-50 infusion and the CT scan procedures. CT scans of the area(s) of overstretch site and representative areas not subjected to overstretch injury were made at the times shown above in Table 4. In addition, whole body CT scans were made at contiguous 5 mm slices (at pretreatment, 4 hours and 8 hour studies).

The computer tomography (CT) imaging was performed with a GE® Hi-Speed Computed Tomography Scanner (Milwaukee, Wis.). The CT images were stored as DICOM3 images in GE® optical disks, CD-Rom. The parameter for CT imaging were 1 mm contiguous slices at 120 kvp, 1.5 seconds at 150 mA. CT scans of the entire rabbit were performed (from rostral nasal area to pelvis at specified times). Iodine uptake within the carotid arteries, vascular, brain and liver tissues were estimated. Contrast attenuation was measured as Hounsfield unites and expressed as “% uptake.” The carotid arteries were evaluated for any contrast uptake in the area where plaques may have formed. After the last CT scan, the rabbits were sacrificed, and the following tissues were preserved in 10% formalin and sent to EPL Associates for histopathological examination: heart, lung, both carotid arteries (including the area subjected to the overstretch injury, and non-treated areas), spleen, liver, lymph nodes, kidneys, and any visible gross lesions as determined during examination.

### B. Results

Clinical observations: Initially, all of the rabbits refused to eat the cholesterol enriched diet, although this was the only food they were offered for three (3) days. On the fourth day, the diet was modified so that it contained a mixture of 75% normal rabbit chow and 25% CED. This mixture was gradually modified over 7 days until only the CED was being fed. The rabbits continued to eat throughout the study. Starting at day 10 post procedure, several rabbits started eating less; small amounts of alfalfa were given along with the CED to help stimulate their appetite. Although the rabbits continued to eat, three of the rabbits had significant weight loss (i.e., <10% body weight). Tables 5 and 6 below demonstrate the weight change and cholesterol levels of the rabbits used in the study.

TABLE 6
Assessment of weight change

| | Weight | Weight | % Weight loss | |
|---|---|---|---|---|
| Rabbit No. | Nov. 20, 2001 | Jan. 18, 2002 | or gain | End Point |
| 3081 | 2.73 | 2.69 | −1.47 | Completed study |
| 3086 | 2.68 | 2.89 | 7.84 | Completed study |
| 3078 | 2.64 | 2.30 | −12.88 | Died day 13 |
| 3083 | 2.59 | 2.76 | 6.56 | Died during |
| | | | | induction |
| 3084 | 2.75 | 2.12 | −22.91 | Died day 14 |
| 3089 | 2.74 | 2.26 | −17.52 | Died day 12 |
| Total | 2.69 ± 0.06 | 2.50 ± 0.32 | −6.73 ± 12.04 | |

TABLE 7
Cholesterol Levels

| Rabbit No. | Pre-treatment* | Dec. 28, 2001 | Jan. 24, 2002 |
|---|---|---|---|
| 3081 | 283 | 2130 | 2830 |
| 3086 | 211 | 2175 | 3860 |
| 3078 | 256 | 2323 | — |
| 3083 | 291 | 4200 | — |
| 3084 | 218 | 2120 | — |
| 3089 | 247 | 2860 | — |

*Normal cholesterol levels are between 200-300

Just prior to the CT scans, blood samples were taken from the rabbits and a routine blood work was performed. Table 7 is a representative sample of the results of this blood work for rabbit numbers 3081 and 3086.

TABLE 8
Blood Assessment

| | | |
|---|---|---|
| Rabbit No. | 3081 | 3086 |

| Hematocrit (Hct) | 22.5 | | 20.5 | |
|---|---|---|---|---|
| Nucleated RBCS | 170 | | 108 | |
| White Blood Cell Ct. (WBC) | 17.3 × 10⁶ | | 94.3 × 10⁶ | |
| Cholesterol | 2830 | mg/dl | 3860 | mg/dl |
| AST | 129 | U/L | 93 | U/L |
| ALT | 81 | U/L | 91 | U/L |
| Glucose | 216 | mg/dl | 185 | mg/dl |
| Phosphorous | 2.8 | mg/dl | 3.8 | mg. dl |

It was determined that the samples “were very lipemic and may affect some of the measurements. Both rabbits had normal plasma and albumin with a regenerative anemia that is likely due to extra-vascular erythrocyte destruction. The ALT levels suggested a chronic liver disease and is likely related to hepatic lipidosis and may also reflect anemia-produced hypoxic injury. The elevated AST suggests liver and possible muscle disease and may also be a reflection of lemolysis.” Extensive review of the literature revealed that rabbits do not tolerate a diet of 2% cholesterol very well, and a level of 0.25%-1% is to recommended (especially for New Zealand Whites). It is believed that the high cholesterol level led to the weight loss and early death of four rabbits. No adverse reactions were noted during the infusion of PH-50. Although the animals were anesthetized, no increase in blood pressure or heart rate was noted.

The primary focus of this study was to study the potential uptake by atherosclerotic plaques of the compound PH-50. The spatial resolution of the CT scanner used in these experiments was approximately 0.15 mm and determination of plaque formation and PH-50 uptake were correlated with histopathology. The timetable for the schedule of CT studies for rabbit numbers 3081 and 3086 are outlined in Table 8.

TABLE 9
CT Schedule

| | | | | Time | |
|---|---|---|---|---|---|
| Exam No. | Series | Description | Time | (post injection) | Images |
| Rabbit No. 3081 | | | | | |
| 345 | 3 | 5 mm survey | — | 5 mm survey | 64 |
| 345 | 4 | 1 mm neck | — | Pre-treatment | 71 |
| 345 | 5 | 1 mm neck | 19:58 | 005 min | 71 |
| 345 | 6 | 1 mm neck | 20:08 | 015 min | 71 |
| 345 | 9 | 1 mm neck | 20:16 | 023 min | 71 |
| 345 | 10 | 1 mm neck | 20:30 | 037 min | 71 |
| 345 | 11 | 1 mm neck | 20:53 | 060 min | 71 |
| 347 | 3 | 5 mm survey | 00:08 | 5 mm survey | 66 |
| 347 | 4 | 1 mm neck | 00:08 | 255 min | 81 |
| 349 | 5 | 5 mm survey | 04:08 | 5 mm survey | 66 |
| 349 | 6 | 1 mm neck | 04:08 | 495 min | 72 |
| 350 | 3 | phantom | n/a | phantom | 13 |
| Rabbit No. 3086 | | | | | |
| 342 | 3 | 5 mm survey | — | 5 mm survey | 58 |
| 342 | 4 | 1 mm neck | — | Pre-treatment | 71 |
| 342 | 5 | 1 mm neck | 16:43 | 005 min | 71 |
| 343 | 2 | 1 mm neck | 17:05 | 005 min | 71 |
| 344 | 1 | 1 mm neck | 17:15 | 015 min | 71 |
| 344 | 2 | 1 mm neck | 17:30 | 030 min | 71 |
| 344 | 3 | 1 mm neck | 18:00 | 060 min | 71 |
| 346 | 3 | 5 mm survey | 21:10 | 5 mm survey | 63 |
| 346 | 4 | 1 mm neck | 21:10 | 250 min | 73 |
| 348 | 3 | 5 mm survey | 01:00 | 5 mm survey | 72 |
| 348 | 4 | 1 mm neck | 01:00 | 480 min | 80 |

The vascular, hepatic, cardiac, liver, spleen and brain uptake data appear below in Table 9.

TABLE 10
Uptake of PH-50 in vascular, hepatic cardiac, liver, spleen and brain tissues

| | | | | | | | |
|---|---|---|---|---|---|---|---|
| Time of | | Posterior | Portal | | | | |
| measurement | Aorta | Vena Cava | Vein | Cardiac | Liver | Spleen | Brain |

Hounsfield Units (HU)

| Rabbit No. 3081 | | | | | | | |
|---|---|---|---|---|---|---|---|
| Pre-treatment | 67.3 | 67.6 | 43.9 | 38.1 | 44.1 | 88.1 | 29.9 |
| 255 min | 56.5 | 95.3 | 56.4 | 136.2 | 169.3 | 449.8 | 49.1 |
| 495 min | 89.3 | 87.8 | 55.4 | 138.8 | 110.6 | 351.2 | 36.8 |
| Rabbit No. 3086 | | | | | | | |
| Pre-treatment | 34.3 | 47.1 | 44.4 | 68.8 | 49.3 | 99.2 | 38.5 |
| 250 min | 64.0 | 96.5 | 54.5 | 220.2 | 189.4 | 786.6 | 31.3 |
| 480 min | 24.1 | 58.6 | 28.1 | 200.3 | 98.2 | 228.5 | 34.7 |

Percent Uptake (%)

| Rabbit No. 3081 | | | | | | | |
|---|---|---|---|---|---|---|---|
| Pre-treatment | 100.0 | 100.0 | 100.0 | 100.0 | 100.0 | 100.0 | 100.0 |
| 255 min | 84.0 | 141.0 | 128.5 | 383.3 | 383.9 | 510.6 | 164.2 |
| 495 min | 132.7 | 129.9 | 126.2 | 250.8 | 250.8 | 398.6 | 123.1 |
| Rabbit No. 3086 | | | | | | | |
| Pre-treatment | 100.0 | 100.0 | 100.0 | 100.0 | 100.0 | 100.0 | 100.0 |
| 250 min | 186.6 | 204.9 | 122.7 | 320.1 | 384.2 | 792.9 | 81.3 |
| 480 min | 70.3 | 124.4 | 63.3 | 291.1 | 199.2 | 230.3 | 90.1 |

All sets of data were carefully harvested, analyzed and then correlated with histopathology. Images were collected, labeled as either “carotid”, “Lung”, “Spleen”, “Liver”, “Cardiac”, “Renal” or “Faxitron images of the carotids,” and then graded according to a Histopathology Correlation Scale (HCS), where 0=no correlation and 5=direct correlation. The results of these experiments are summarized below:

Carotid images: Images of the carotid tissues taken at 30 minutes showed vascular structures such as the jugular vein and carotid artery. At this time point, the plaques were not visible due to the amount of contrast in the lumen of the vessels. However, a carotid plaque was visible after 250 minutes. An HCS score of 2 was assigned due to the minimal contrast uptake in the left carotid of rabbit no. 3086.

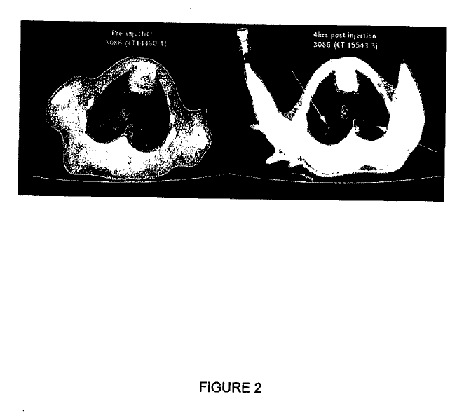

Lung images: Pre-injection images of the lung tissue showed a uniform lung pattern, with vascular structures and no contrast. However, after 4 hours post-injection, uptake in the vascular in at least two locations was visible. An HCS score of 4 assigned because contrast uptake in the small arterioles of the lung of rabbit no. 2086 was visible (FIG. 2).

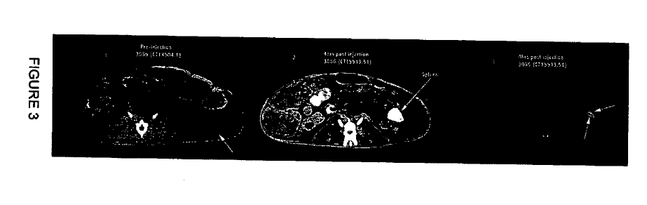

Spleen images: Pre-injection images of the spleen tissue showed uniform density. At 4 hours post-injection, the density of the spleen was greatly increased, with evidence of uptake within the splenic vascular structures. An HCS score of 4 was assigned due to the contrast uptake in some of the small arterioles of the spleen of rabbit no. 3086 (FIG. 3).

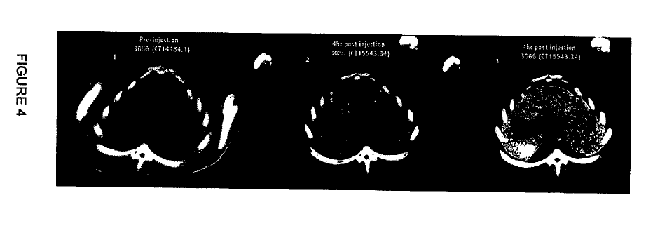

Liver images: Pre-injection images of the hepatic tissues also showed uniform density. At 4 hours post-injection, there appeared to be uniform uptake with non-homogenous density, with the vascular structures showing enhanced contrast. An HCS score of 3 was assigned because there was a modeled appearance to the hepatic parenchyma which was believed to indicate contrast uptake in some of the small arterioles of the liver of rabbit no. 3086 (FIG. 4).

Cardiac images: Pre-injection cardiac tissues appeared uniform in density with the chambers being barely visible. At 4 hours post-injection, there appeared to be definitive uptake in the are of the coronary arteries. At 8 hours post-injection, there appeared to be continued uptake in the area of the coronary arteries. An HCS score of 4 was assigned because of the indicated contrast uptake in plaques in the coronary arteries and myocardium of rabbit no. 3086.

Renal images: At 4 hours post-injection, the images showed a dense structure in the right renal artery which was not visible in the pre-injection image, demonstrating contrast uptake in the renal artery. An HCS score of 0 was assigned because although contrast uptake was found in the renal artery of rabbit no. 3086, this area was not submitted for histopathology.

Faxitron images: The only image which appeared to show any contrast uptake in the area of injury in rabbit no. 3086 was the left carotid artery, which correlated well with the CT and histopathology results. An HCS score of 3 was assigned because of the detected contrast uptake in the mid region of the left carotid artery of rabbit no. 3086.

All harvested tissues appeared normal, except for the liver, which was pale in color and very friable. All of the rabbits examined had a similar appearance. Samples were also sent to a pathologist for further analysis.

### C. Conclusions

The preliminary examination of the percent increase in contrast (as compared to the pre-injection values) as performed in these experiments did not allow for the optimal time for imaging to be determined. However, it does appear that plaque uptake can be seen on the 4 and 8 hour infusion scans.

The vascular structures were well visualized at fifteen (15) minutes post-infusion and for the remainder of the ninety (90) minute infusion. The infusion of a high dose also gave excellent cardiac and hepatic images, in addition to vascular visualization.

The results demonstrate that the iodinated nanoparticle can be used effectively and efficiently for CT vascular imaging studies. PH-50 also appeared to have been taken up by atherosclerotic plaques in the heart and lung.

The overstretch injury did not produce the extent of atherosclerotic plaques intended. However, spontaneous atherosclerotic plaques did appear in several areas. The image data correlated well with the histopathology, showing the plaques and uptake of PH-50 by atherosclerotic plaques.

The data further indicate that PH-50 appears to be effective to determining the presence of atherosclerotic plaques.

### Example 3

### Planned Clinical Development: Protocol Outline

A. Objective

The objective of this study will be for the preliminary assessment of tolerability of PH-50 after intravenous administration at dose levels (e.g., 0.1 to 4 ml/kg based on a 150 mg/ml stock) and administration rates (e.g., 0.6 to 1.2 ml/sec), which are anticipated to be used from x-ray computed tomographic assessment of cardiovascular diseases, such as peripheral vascular disease, coronary artery disease and carotid artery disease. The evaluation of efficacy (dose-related vascular opacification) after intravenous administration will be used for development of dose-ranging studies (see Example 4 below) in patients with selected cardiovascular diseases. The measurement of blood levels and urinary recovery of total iodine, parent drug and free-acid hydrolysis products (including conjugates) after intravenous administration will be performed.

B. Study Design

The study will be observational and baseline-controlled (within-patient), unrandomized, open-label ascending-dose, parallel-group, single-center.

C. Materials

A total of up to 36 healthy, nonsmoking volunteers as assessed by medical history, current physical examination, clinical laboratory testing, electrocardiography, and pulmonary-function and pulse-oximetry testing will be selected provided that (1) the results for all clinical laboratory tests for liver function or injury do no exceed laboratory reference range; (2) the results for pulmonary function test are not below-aged-adjusted normal-range values; and (3) the volunteer is not at the early stages of, in the middle of, or convalescing from any systemic infectious disease, including the common cold or any nonsystemic infectious process with the exception of ordinary dermal or mucosal fungal infections that can be treated with topical nonprescription antifungal preparations. Furthermore, the subjects will be adults within the age range of 18 to 64 years, of either sex, provided that women of childbearing potential employ effective contraceptive methods and test negative for urinary 1-human chorionic gonadotropin within 72 hours prior to drug administration and willingly and knowingly consent to participate in the study in a manner that complies with current FDA regulations.

Control conditions will comprise a negative control based on the baseline (pretreatment) observations taken between enrollment and the start of PH-50 administration.

D. Methods

The PH-50 compound will be administered between 7:00 and 10:00 am after an overnight fast. No caffeine-containing beverages or sugar-containing beverages will be administered prior to the first serum chemistry blood sample post administration (see below). A light meal may be taken thereafter and normal meal routine resumed at option of the volunteer four hours post-administration.

The dose levels and administration rates will be in twelve groups of three volunteers each, with at least one of each sex per group. The proposed dose and administration-rate groups are shown below in Table 11.

TABLE 11
Proposed Dosage and Administration-Rates

| Dose (mL/Kg) | Administration Rate (mL/sec) |
|---|---|
| 0.1 | 0.6 |
| 0.1 | 1.2 |
| 0.25 | 0.6 |
| 0.25 | 1.2 |
| 0.5 | 0.6 |
| 0.5 | 1.2 |
| 1.0 | 0.6 |
| 1.0 | 1.2 |
| 2.0 | 0.6 |
| 2.0 | 1.2 |
| 4.0 | 0.6 |
| 4.0 | 1.2 |

During the course of drug administration, the following observations will be assessed: vital signs, including respiratory rate and oral temperature, heart rate and blood pressure. Electrocardiography and pulse oximetry and pulmonary function testing for oxygen saturation will be performed. Hemogram and leukogram (absolute, not relative/differential count) and platelet counts will be assessed. Serum chemistry (e.g., SMAC-23 or equivalent panels, including but not limited to albumin, alkaline phosphatase, calcium, carbon dioxide, chloride, cholesterol, creatine phosphokinase (CPK, creatine kinase [CK]), creatinine, direct (unconjugated) bilirubin, gamma-glutamyltransferase (GGT), glucose, inorganic phosphorous, lactate (lactic) dehydrogenase (LDM), potassium, serum glutamic oxaloacetic transaminase (SGOT, aspartate aminotransferase [AST]), serum glutamic pyruvic transaminase (SGPT, alanine aminotransferase [ALT]), sodium, triglycerides, total bilirubin, total protein, urea nitrogen (BUN), and uric acid) will be measured. Furthermore, the potential for complement activation will be monitored by functional assay of total hemolytic complement (CH50) in blood serum samples taken at the same times as for the other serum chemistry assays. Urinalysis will also be evaluated as well as the plasma concentrations of parent drug substance and major metabolite(s). Urinary recovery and excretion rates of the parent drug and metabolite(s) will be evaluated by LC/MS.

Radiography will be performed prior to administration and at 20 and 40 minutes post-administration (limited by total radiation absorbed dose to skin) by a x-ray tomograph (CT) with settings optimum for the vascular bed of interest. The radiopacity in the regions of interest drawn within the major vessels (e.g., aorta, renal, carotid, peripheral, cerebral vessels), other vessels and tissues contained in representative slices will be measured at each observation interval and expressed in terms of Hounsfield units (HUs). In both pre- and post-administration images, an external standard will be positioned within the imaging volume.

### Example 4

### Planned Clinical Development: Protocol Outline

A. Objective

The primary objective of this clinical study will be to investigate and determine the minimum effective and optimal intravenous dose of PH-50, including administration rate, required to provide an effective level of vascular contrast enhancement that will facilitate the identification, characterization, and determination of the severity of vascular disease during spiral (helical) computed tomography imaging of the aorta, renal, peripheral and carotid arteries. The starting dose, dose rate, and incremental doses will be determined from and based in the results obtained from the tolerance study (described in Example 3).

Secondary objectives of this proposed study will include an expanded assessment of the imaging equipment, including CT imaging parameters, that provide effective visualization and contrast enhancement of abnormalities observed in vascular regions characterized by either turbulent flow, low flow, or organ perfusion such as the kidneys. These studies will rely on the imaging experience gained during the preliminary image assessment of the PH-50 tolerance studies in normal human volunteers.

B. Study Design and Methodology

This study will be a controlled, comparative study in patients scheduled to undergo angiographic assessment of vascular disease in a defined vascular region. This will include enrollment of patients that are suspected to have vascular disease in the aorta or the renal, carotid, iliac, femoral or peripheral arteries. Patients will be randomized to undergo the angiography examination, using an approved iodinated contrast media, before or after the PH-50 CT examination. All patients will undergo a non-contrast CT examination that will include, but not be limited to, the vascular region scheduled to be examined by angiography. A PH-50 contrast examination will be performed immediately following the non-contrast CT using the same equipment and settings and vascular beds as the non-contrast CT exam. Both the non-contrast and contrast CT exams will include the vascular bed in the region of clinical interest, but may also include the vasculature that may be readily imaged by CT that are adjacent to or outside of the clinical region of interest.

C. Dosing

This study will be an escalating dose study, with the starting dose for PH-50 based on the efficacy and tolerance results collected during the above study (see Example 3).

The interpretation of the angiographic evaluation will be regarded as standard of truth. Both the non-contrast CT and PH-50 contrast enhanced CT images will be compared with the standard of truth. The location, size, percent stenosis, and other distinguishing features interpreted from the non-contrast and PH-50 contrast enhanced CT studies will be compared to the standard of truth. All images will be interpreted by a blinded, independent panel of expert radiologists.

D. Diagnosis and Main Criteria for Inclusion/Exclusion

A total of up to 100 patients may be enrolled in order to obtain 80 valuable patients. Baseline medical history, physical examination, clinical laboratory testing, electrocardiography, pulse oximetry and pulmonary-function testing will be obtained.

E. Efficacy

The following comparative assessments will be used to assess the clinical effectiveness of the PH-50 contrast enhanced CT images: (1) using the angiography examination as the gold standard, the non-contrast enhanced CT and PH-50 contrast enhanced CT examinations will be evaluated in a randomized manner by a panel of blinded, independent qualified readers to assess the number, location, severity, and distinguishing (descriptive) characteristics of each lesion. These findings will be compared to the gold standard angiographic findings on a lesion by lesion, vascular bed and patient-by-patient basis; (B) because gold standard information will only be obtained from the vascular beds of clinical interest, and adjacent vascular beds may also be evaluated by non-contrast and PH-50 enhanced CT, the nature and characteristics of vascular abnormalities observed in these adjacent vascular beds will also be recorded in order to determine if the abnormalities are limited to the vascular bed of clinical interest or are global.

F. Drug Administration

PH-50 will be administered following completion of the non-enhanced CT images. Dose levels, administration rates, and group size will be determined after the results of the tolerability and preliminary efficacy study are analyzed. Patients may be withdrawn from the study if they experience treatment related adverse events defined as follows: (a) a serious adverse event that is reasonably related to PH-50 or to its administration; (b) withdrawal of the subject from the study (withdrawal of informed consent after drug administration) for any adverse event; and (c) appearance of a characteristic symptom of an incipient serious adverse event or of a characteristic sign of an occult serious adverse event that may be reasonably related to the drug or to its administration.

G. Observations and Evaluation

Treatment-emergent signs and symptoms of drug toxicity will be monitored continuously throughout drug administration and for one hour afterward, and at intervals at which other observations (below) are scheduled. The total observation interval will be assigned after reviewing the findings of the completed clinical trial.

The following observations will be made as follows: vital signs, such as heart rate, systolic and diastolic blood pressure, respiratory rate and oral temperature. Urinalysis will also be measured. Heart rate and 12-lead electrocardiogram will be recorded just prior to beginning of drug administration, throughout drug administration and continuing for five minutes afterward for 5-sec epochs every 30 sec, and at 10, 15, 20, 40, 60 and 90 min, and at 3, 6, 12 and 24 and 72 hours. Pulmonary function testing will be assessed by oxygen saturation at same observation time points as for vital signs. Forced expiratory volume in one second (FEV₁) and forced vital capacity (FVC) will be measured prior to drug administration and at 20 min, and 1 and 24 and 72 h post-administration. Continuous oxygen saturation monitoring (pulse oximetry) will be measured starting just prior to beginning of drug administration and continuing throughout drug administration imaging, then again at the same observation time points as for vital signs. Complete blood count (CBC) with differential count (absolute, not relative), and platelet count—once between enrollment and beginning of drug administration (actual time at liberty within that window, but accurately recorded), will be evaluated at 2, 12, 24 and 72 hours post-administration. Serum chemistry (e.g., SMAC-23 or equivalent panels, including but not limited to albumin, alkaline phosphatase, calcium, carbon dioxide, chloride, cholesterol, creatine phosphokinase (CPK, creatine kinase [CK]), creatinine, direct (unconjugated) bilirubin, gamma-glutamyltransferase (GGT), glucose, inorganic phosphorous, lactate (lactic) dehydrogenase (LDH), potassium, serum glutamic oxaloacetic transaminase (SGOT, aspartate aminotransferase [AST]), serum glutamic pyruvic transaminase (SGPT, alanine aminotransferase [ALT]), sodium, triglycerides, total bilirubin, total protein, urea nitrogen (BUN), and uric acid) will be evaluated once between enrollment and beginning of drug administration, at 2, 12, 24 and 72 hours after dosing.

CT Examinations will be performed before study drug administration, and at 20 min and 40 min after (limited by total radiation absorbed dose to skin) by a x-ray tomograph (CT) with settings optimum for the vascular bed of interest; the radiopacity in regions of interest drawn within the major vessels (e.g., aorta, renal, carotid, peripheral) other vessels and tissues contained in representative slices will be evaluated at each observation interval. The images will be assessed for the presence, location, size, severity, and descriptive characteristics of abnormalities in the vascular bed of clinical interest, as well as adjacent vascular beds. Radiopacity in regions of interest will be measured by the imaging core lab used to coordinate the blinded image review and reported as Hounsfield units (HUs).

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1-41. (canceled)

42. A method for obtaining an image of vulnerable vascular plaque, if present in a human subject, the method comprising:
a) administering to a the human subject the nanoparticulate contrast agent 6-ethoxy-6-oxohexy-3,5-bis(acetylamino)-2,4,6-triiodobenzoate, wherein the contrast agent is administered in an amount sufficient to be taken up by macrophages accumulated in the vulnerable vascular plaque; and
b) detecting the contrast agent in the macrophages and thereby obtaining an image of the vulnerable vascular plaque in the human subject.

43. The method of claim 42, wherein the mean particle size of the nanoparticulate contrast agent is from about 20 nanometers to about 750 nanometers.

44. The method of claim 42, wherein the mean particle size of the nanoparticulate contrast agent is from about 200 nanometers to about 400 nanometers.

45. The method of claim 42, wherein the mean particle size of the nanoparticulate contrast agent is about 300 nanometers.

46. The method of claim 42, wherein the contrast agent is administered parenterally.

47. The method of claim 42, wherein the contrast agent is administered intravenously.

48. The method of claim 42, wherein the step of detecting comprises using a method selected from the group consisting of x-ray imaging, ultrasonography, computed tomography, computed tomography angiography, electron beam, magnetic resonance imaging, magnetic resonance angiography, and positron emission tomography.

49. The method of claim 42 wherein the contrast agent is administered intravenously or intrarterially and the detecting step comprises measuring the radiodensity of the macrophages using computed tomography (CT).

50. The method of claim 42, wherein the detecting step comprises generating multiple images or a series of images of the contrast agent in the macrophages.

51. The method of claim 42, further comprising predicting whether the subject is at risk for a vascular disease.

52. The method of claim 51, wherein the vascular disease is selected from the group consisting of atherosclerosis, coronary artery disease (CAD), myocardial infarction (MI), ischemia, stroke, peripheral vascular diseases, and venous thromboembolism.

53. The method of claim 42, wherein the contrast agent is administered at more than one time point.

54. The method of claim 42, wherein the contrast agent is administered by infusion over a period of about five minutes or more.

55. The method of claim 42, wherein detecting the contrast agent occurs at least about 30 minutes or more after administration of the contrast agent.

56. The method of claim 42, wherein detecting the contrast agent occurs at least about 1 hour or more after administration of the contrast agent.

57. The method of claim 42, wherein detecting the contrast agent occurs at least about 2 hours or more after administration of the contrast agent.

58. The method of claim 42, wherein detecting the contrast agent occurs at least about 9 hours or more after administration of the contrast agent.

59. The method of claim 42, wherein the contrast agent is administered at a dose of about 0.1 mL/kg to about 8 mL/kg, based on a stock solution of about 15% weight/volume.

60. The method of claim 42, wherein the contrast agent has a mean particle size of less than about 300 nm.

61. A method for diagnosing atherosclerosis in a human subject, the method comprising:
a) examining an image for the presence or absence of vascular plaque, wherein the image is obtained by:
i. administering to the human subject the nanoparticulate contrast agent 6-ethoxy-6-oxohexy-3,5-bis(acetylamino)-2,4,6-triiodobenzoate, wherein the contrast agent is administered in an amount sufficient to be taken up by macrophages that accumulate in vascular plaque; and
ii. detecting the contrast agent taken up by the macrophages, and
b) concluding whether the detecting step has revealed vascular plaque, wherein the presence of vascular plaque is indicative of atherosclerosis and thereby supports a diagnosis of atherosclerosis in the human subject.

62. The method of claim 42 or claim 61, wherein the contrast agent is present in a formulation comprising a poloxamer and polyethylene glycol.

63. The method of claim 62, wherein the contrast agent is present in a formulation comprising poloxamer 338 and polyethylene glycol 1450.

64. The method of claim 42 or claim 61, wherein at least 50% of the nanoparticles of the nanoparticulate contrast agent have a diameter of not more than about 350 mm.

65. The method of claim 42 or claim 61, wherein the administering step and the detecting step are carried out from about 0.5 to about 9 hours apart.
